(19) United States
(12) Patent Application Publication
(10) Pub. No.: US 2025/0095513 A1
(43) Pub. Date: Mar. 20, 2025

(54) METHODS AND SYSTEMS FOR SIMULATING A DYNAMIC FLUORESCENCE RESPONSE

(71) Applicant: Stryker Corporation, Portage, MI (US)

(72) Inventors: Robert Anthony STEAD, North Vancouver (CA); Claire Rosemarie Leeann FORBES, Vancouver (CA); Thomas Darryl NEWTON, Vancouver (CA); Marc ANDRÈ, Spiegel b. Bern (CH); John Josef Paul FENGLER, North Vancouver (CA)

(73) Assignee: Stryker Corporation, Portage, MI (US)

(21) Appl. No.: 18/888,720

(22) Filed: Sep. 18, 2024

Related U.S. Application Data
(60) Provisional application No. 63/583,855, filed on Sep. 19, 2023.

(51) Int. Cl. G09B 23/28; A61B 5/00; G01N 21/64

(52) U.S. Cl. CPC G09B 23/286; G01N 21/6408; G01N 21/6428; G01N 21/6456; A61B 5/0071; G01N 2021/6439; G01N 2201/062; G09B 23/285

(57) ABSTRACT

The present disclosure relates generally to medical imaging, and more specifically to devices and systems for simulating a time-varying fluorescence response. An exemplary device for simulating a fluorescence response of a tissue comprises: a simulated tissue layer; one or more optical detectors to detect a fluorescence excitation light incident on the simulated tissue layer; a plurality of illumination sources underneath the simulated tissue layer; and a controller configured to control the plurality of illumination sources to emit light based on the detected fluorescence excitation light and at least one time-varying pattern.

### CROSS-REFERENCE TO RELATED APPLICATION

This application claims the benefit of U.S. Provisional Application 63/583,855 filed on Sep. 19, 2023, the entire content of which is incorporated herein by reference for all purposes.

### FIELD

The present disclosure relates generally to medical imaging, and more specifically to devices and systems for simulating a time-varying fluorescence response.

### BACKGROUND

Existing simulation systems and devices can be used to mimic properties of organic tissue (e.g., of a human subject) for the purpose of testing and demonstrating fluorescence imaging systems. For example, when illuminated by a fluorescence excitation light, a simulation device can be configured to generate a fluorescence response, which can be captured by fluorescence imaging systems. Existing simulation devices or systems use either static materials (e.g., passively excited light-emitting diodes or LEDs, passively excited materials) or fluorescent liquids contained in various structures (e.g., pipes, tubing, channels) to simulate a fluorescence response.

However, many existing simulation devices cannot simulate important characteristics of organic tissue, such as the time-dependent nature of certain fluorescence responses. For common applications of fluorescence imaging for perfusion assessment, the clinical use of the fluorescence imaging system involves obtaining a video or a time series of images of the fluorescence response of the tissue following injection of the fluorescent contrast agent. The tissue exhibits a fluorescence response starting from zero prior to injection of the contrast agent, increasing as the contrast agent arrives and is distributed throughout the scene, and followed by the subsequent decay of the fluorescence response as the contrast agent is metabolized or broken down by the body and removed from the scene. However, static materials are designed to reproduce the brightness of a particular clinical environment at a particular instant in time and cannot reproduce the time-dependent dynamics. Thus, these simulation devices have limited utility because they cannot be used to demonstrate to users how to make clinical decisions based on the time-varying data they would observe in an actual use case or to test products that analyze the dynamic fluorescence signals (e.g., to identify healthy perfusion v. abnormal perfusion).

Further, many existing simulation devices are not robust and require regular maintenance. For example, some simulation devices involve a liquid fluorescence contrast agent that is pumped through and circulates within an assembly or structure containing tubing or ducts. These devices require a system of tubing to contain the liquid contrast agent and can be susceptible to leaks of the liquid contrast agent. Further, the fluorescence of the liquid can decay over long time periods due to, for example, photobleaching and dimerization. Thus, the liquid must be replaced periodically in order to maintain a consistent fluorescence response of the device.

Thus, it is desirable to provide devices that can simulate a dynamic (i.e., time-varying) fluorescence response that is also robust, reliant, and easy to maintain.

### SUMMARY

Disclosed herein are exemplary devices, apparatuses, systems, methods, and non-transitory storage media for simulating a fluorescence response of a tissue (e.g., organic tissue of a human subject). The techniques described herein can provide a device mimicking the clinically relevant properties of a tissue in a dynamic manner to test and demonstrate fluorescence imaging systems. In particular, the device can simulate the time-dependent nature of a fluorescence response that results from, for example, the temporal dynamics of the fluorophore level in the tissue for perfusion assessment. Further, the techniques described herein can allow a user to modify the fluorescence characteristics of the device via modification of only a software component, thereby avoiding the need to make hardware modifications to simulate additional fluorescent applications subsequent to assembly of the hardware.

An exemplary device can comprise a simulated tissue layer and one or more optical detectors to detect a fluorescence excitation light incident on the simulated tissue layer. The device can further comprise a plurality of illumination sources underneath the simulated tissue layer and a controller configured to control the plurality of illumination sources to emit light based on the detected fluorescence excitation light and at least one time-varying pattern. By controlling the illumination sources, the device can control the amount of emitted light based on a time-varying pattern (e.g., as defined by a function of time). Because the illumination sources are arranged in a formation (e.g., an array), the amount of simulated fluorescence light emitted can be varied spatially to simulate different anatomies having varying rates of fluorophore uptake.

The techniques described herein provide numerous technical advantages. The device can mimic the clinically relevant properties (e.g., optical properties, physical properties) of a tissue in a dynamic manner to test and demonstrate fluorescence imaging systems. The device can allow the magnitude (i.e., brightness) of the fluorescence response to be varied both spatially and temporally in order to replicate a desirable fluorescence response. The emitted light of the fluorescence response does not result from optical excitation of the illumination sources, but rather is controlled by modulating the electrical current to the illumination sources, thus resulting in a more reliable and more consistent system. Further, the techniques described herein can allow a user to modify the fluorescence characteristics of the device via modification of only a software component, thereby avoiding the need to make hardware modifications to simulate additional fluorescent applications subsequent to assembly of the hardware. Further, the device is composed of solid-state components having high reliability and a long lifetime, thus producing a consistent fluorescence response over many uses, regardless of the frequency of use.

An exemplary device for simulating a fluorescence response of a tissue comprises: a simulated tissue layer; one or more optical detectors to detect a fluorescence excitation light incident on the simulated tissue layer; a plurality of illumination sources underneath the simulated tissue layer; and a controller configured to control the plurality of illumination sources to emit light based on the detected fluorescence excitation light and at least one time-varying pattern.

The fluorescence excitation light may include an infrared light, a visible light, or an ultraviolet light. The emitted light by the plurality of illumination sources may include an infrared light, a visible light, or an ultraviolet light. Controlling the plurality of illumination sources to emit light may include: determining if the magnitude of the fluorescence excitation light exceeds a threshold; in accordance with a determination that the magnitude of the fluorescence excitation light exceeds the threshold, turning on the plurality of illumination sources; and in accordance with a determination that the magnitude of the fluorescence excitation light does not exceed the threshold, turning off the plurality of illumination sources. The threshold may be set as an absolute value or as a relative value of the detected fluorescence excitation light during an on time. The controller may be configured to determine the magnitude of the emitted light based on the magnitude of the fluorescence excitation light incident on the simulated tissue layer of the device. The magnitude of the emitted light may be proportional to the magnitude of the fluorescence excitation light.

The at least one time-varying pattern may include pulsing between a first intensity level and a second intensity level lower than the first. The at least one time-varying pattern may include a fluorescence growth and decay function. The at least one fluorescence growth and decay function may include one or more parameters for simulating ingress and egress of a fluorescent contrast agent at the tissue over time. The plurality of illumination sources may include a first illumination source and a second illumination source, the at least one time-varying pattern includes a first time-varying pattern and a second time-varying pattern; and controlling the plurality of illumination sources of the device to emit light further includes: controlling the first illumination source based on the first time-varying pattern; and controlling the second illumination source based on the second time-varying pattern. The at least one time-varying pattern may be based on a file comprising a plurality of fluorescence image frames. The file may be artificially generated or generated based on a set of fluorescence video data.

The simulated tissue layer may include silicone. The one or more optical detectors may include one or more photodiodes. The plurality of illumination sources may include a plurality of light-emitting diodes (LEDs). The device may further include one or more spectral filters over the one or more optical detectors. The device may further include one or more long pass dichroic filters, one or more light pipes, or any combination thereof.

The controller may include one or more PWM controllers for controlling the plurality of illumination sources. The controller may include an adjustable current or voltage source to drive the plurality of illumination sources. The controller may include a programmable device for controlling the plurality of illumination sources. The device may further include a data port for remote control. The device may be wirelessly controlled. Controlling the plurality of illumination sources of the device to emit light may be further based on one or more user inputs. The one or more user inputs may include one or more button presses, one or more gestural inputs, one or more textual inputs, one or more auditory inputs, one or more selections, or any combination thereof.

An exemplary method for simulating a fluorescence response of a tissue comprises: detecting, using one or more optical detectors, a fluorescence excitation light incident on a simulated tissue layer; and controlling, using a controller, a plurality of illumination sources underneath the simulated tissue layer to emit light based on the detected fluorescence excitation light and at least one time-varying pattern.

The fluorescence excitation light may include an infrared light, a visible light, or an ultraviolet light. The emitted light by the plurality of illumination sources may include an infrared light, a visible light, or an ultraviolet light. Controlling the plurality of illumination sources to emit light may include: determining if the magnitude of the fluorescence excitation light exceeds a threshold; in accordance with a determination that the magnitude of the fluorescence excitation light exceeds the threshold, turning on the plurality of illumination sources; and in accordance with a determination that the magnitude of the fluorescence excitation light does not exceed the threshold, foregoing turning on the plurality of illumination sources. The threshold may be set as an absolute value or as a relative value of the detected fluorescence excitation light during an on time. The method may further include determining the magnitude of the emitted light based on the magnitude of the fluorescence excitation light incident on the simulated tissue layer. The magnitude of the emitted light may be at least partially based on an instantaneous value and a maximum value of the magnitude of the fluorescence excitation light.

The at least one time-varying pattern may include pulsing between a first intensity level and a second intensity level lower than the first. The at least one time-varying pattern may include a fluorescence growth and decay function. The at least one fluorescence growth and decay function may include one or more parameters for simulating ingress and egress of a fluorescent contrast agent at the tissue over time. The plurality of illumination sources may include a first illumination source and a second illumination source, the at least one time-varying pattern includes a first time-varying pattern and a second time-varying pattern; and controlling the plurality of illumination sources of the method to emit light further includes: controlling the first illumination source based on the first time-varying pattern; and controlling the second illumination source based on the second time-varying pattern. The at least one time-varying pattern may be based on a file comprising a plurality of fluorescence image frames. The file may be artificially generated or generated based on a set of fluorescence video data.

The simulated tissue layer may include silicone. The one or more optical detectors may include one or more photodiodes. The plurality of illumination sources may include a plurality of light-emitting diodes (LEDs). The one or more optical detectors may be underneath one or more spectral filters.

The controller may include one or more PWM controllers for controlling the plurality of illumination sources. The controller may include an adjustable current or voltage source to drive the plurality of illumination sources. The controller may include a programmable method for controlling the plurality of illumination sources. Controlling the plurality of illumination sources of the method to emit light may be further based on one or more user inputs. The one or more user inputs may include one or more button presses, one or more gestural inputs, one or more textual inputs, one or more auditory inputs, one or more selections, or any combination thereof.

An exemplary non-transitory computer-readable storage medium stores one or more programs, the one or more programs comprising instructions, which, when executed by one or more processors of an electronic device, cause the electronic device to perform any of the techniques described herein.

### BRIEF DESCRIPTION OF THE FIGURES

The invention will now be described, by way of example only, with reference to the accompanying drawings, in which:

FIG. 1A is an illustration of an endoscopic camera system.

FIG. 1B is a diagram of a portion of the endoscopic camera system of FIG. 1A and a target object for imaging.

FIG. 2 illustrates a schematic view of a system for illumination and imaging.

FIG. 3 is a block diagram of an imaging system.

FIG. 4A illustrates an exemplary device for simulating a fluorescence response of a vascular tissue.

FIG. 4B illustrates an exemplary device for simulating a fluorescence response of a lymphatic tissue.

FIG. 5 illustrates an exemplary process for simulating a fluorescence response of a tissue.

FIG. 6 illustrates an exemplary diagram demonstrating a possible relationship between the magnitude of a fluorescence excitation light and the magnitude of a light emitted by one or more illuminations sources of a simulation device, in accordance with some examples.

FIG. 7 illustrates a fluorescence response produced by an exemplary device implemented according to the techniques described herein, imaged by a fluorescence imaging system.

### DETAILED DESCRIPTION

Reference will now be made in detail to implementations and various aspects and variations of systems and methods described herein. Although several exemplary variations of the systems and methods are described herein, other variations of the systems and methods may include aspects of the systems and methods described herein combined in any suitable manner having combinations of all or some of the aspects described. Examples will now be described more fully hereinafter with reference to the accompanying drawings; however, they may be embodied in different forms and should not be construed as limited to the examples set forth herein. Rather, these examples are provided so that this disclosure will be thorough and complete, and will fully convey exemplary implementations to those skilled in the art.

Disclosed herein are exemplary devices, apparatuses, systems, methods, and non-transitory storage media for simulating a fluorescence response of a tissue (e.g., organic tissue of a human subject). The techniques described herein can provide a device mimicking the clinically relevant properties of a tissue in a dynamic manner to test and demonstrate fluorescence imaging systems. In particular, the device can simulate the time-dependent nature of a fluorescence response that results from, for example, the temporal dynamics of the fluorophore level in the tissue for perfusion assessment. Further, the techniques described herein can allow a user to modify the fluorescence characteristics of the device via modification of only a software component, thereby avoiding the need to make hardware modifications to simulate additional fluorescent applications subsequent to assembly of the hardware.

An exemplary device can comprise a simulated tissue layer and one or more optical detectors to detect a fluorescence excitation light incident on the simulated tissue layer. The device can further comprise a plurality of illumination sources underneath the simulated tissue layer and a controller configured to control the plurality of illumination sources to emit light based on the detected fluorescence excitation light and at least one time-varying pattern. By controlling the illumination sources, the device can control the amount of emitted light based on a time-varying pattern (e.g., as defined by a function of time). Because the illumination sources are arranged in a formation (e.g., an array), the amount of simulated fluorescence light emitted can be varied spatially to simulate different anatomies having varying rates of fluorophore uptake. In some examples, in addition to providing the option to simulate a time-varying fluorescence response as described herein, the device provides another option to simulate a static fluorescence response having a constant magnitude over a period of time. The constant magnitude can be specified by a user. For example, the device can be configured to simulate a first static fluorescence response having a first constant magnitude over a first time period and simulate a second static fluorescence response having a second constant magnitude over a second time period. In some examples, the constant magnitude can include the maximum magnitude that the device can provide.

The techniques described herein provide numerous technical advantages. The device can mimic the clinically relevant properties (e.g., optical properties, physical properties) of a tissue in a dynamic manner to test and demonstrate fluorescence imaging systems. The device can allow the magnitude (i.e., brightness) of the fluorescence response to be varied both spatially and temporally in order to replicate a desirable fluorescence response. The emitted light of the fluorescence response does not result from optical excitation of the illumination sources, but rather is controlled by modulating the electrical current to the illumination sources, thus resulting in a more reliable and more consistent system. Further, the techniques described herein can allow a user to modify the fluorescence characteristics of the device via modification of only a software component, thereby avoiding the need to make hardware modifications to simulate additional fluorescent applications subsequent to assembly of the hardware. Further, the device is composed of solid-state components having high reliability and a long lifetime, thus producing a consistent fluorescence response over many uses, regardless of the frequency of use. Further, as described herein, the simulated tissue layer of the device can be removed and replaced with a different type of simulated tissue layer. In other words, the device can operate with different types of simulated tissue layers (e.g., corresponding to different types of tissues, different organs, different vascular structures) to simulate fluorescence responses from different parts of the anatomy.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general-purpose computer selectively activated or reconfigured by a computer program stored in the computer. Such a computer program may be stored in a non-transitory, computer-readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application-specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

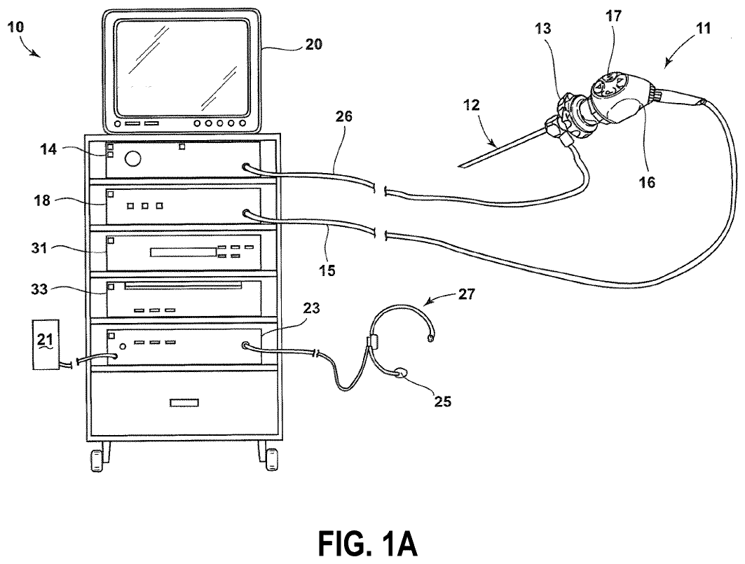

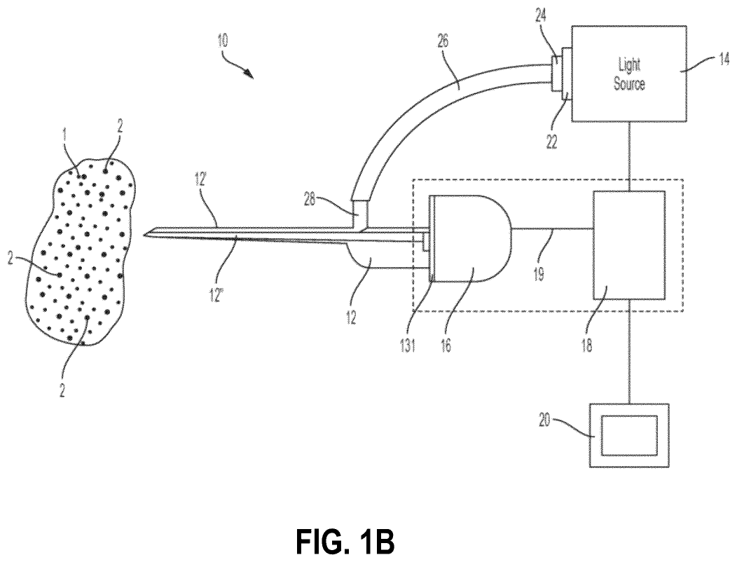

FIG. 1A shows an example of an endoscopic imaging system 10, which includes a scope assembly 11 which may be utilized in endoscopic procedures. The scope assembly 11 incorporates an endoscope or scope 12 which is coupled to a camera head 16 by a coupler 13 located at the distal end of the camera head 16. Light is provided to the scope by a light source 14 via a light guide 26, such as a fiber optic cable. The camera head 16 is coupled to a camera control unit (CCU) 18 by an electrical cable 15. The CCU 18 is connected to, and communicates with, the light source 14. Operation of the camera 16 is controlled, in part, by the CCU 18. The cable 15 conveys video image and/or still image data from the camera head 16 to the CCU 18 and may convey various control signals bi-directionally between the camera head 16 and the CCU 18.

A control or switch arrangement 17 may be provided on the camera head 16 for allowing a user to manually control various functions of the system 10, which may include switching from one imaging mode to another, as discussed further below. Voice commands may be input into a microphone 25 mounted on a headset 27 worn by the practitioner and coupled to the voice-control unit 23. A hand-held control device 29, such as a tablet with a touch screen user interface or a PDA, may be coupled to the voice control unit 23 as a further control interface. In the illustrated example, a recorder 31 and a printer 33 are also coupled to the CCU 18. Additional devices, such as an image capture and archiving device, may be included in the system 10 and coupled to the CCU 18. Video image data acquired by the camera head 16 and processed by the CCU 18 is converted to images, which can be displayed on a monitor 20, recorded by recorder 31, and/or used to generate static images, hard copies of which can be produced by the printer 33.

FIG. 1B shows an example of a portion of the endoscopic system 10 being used to illuminate and receive light from an object 1, such as a surgical site of a patient. The object 1 may include fluorescent markers 2, for example, as a result of the patient being administered a fluorescence imaging agent. The fluorescent markers 2 may comprise, for example, indocyanine green (ICG).

The light source 14 can generate visible illumination light (such as any combination of red, green, and blue light) for generating visible (e.g., white light) images of the target object 1 and, in some examples, can also produce fluorescence excitation illumination light for exciting the fluorescent markers 2 in the target object for generating fluorescence images. In some examples, the light source 14 can produce fluorescence excitation illumination light for exciting autofluorescence in the target object for generating fluorescence images, additionally or alternatively to light for exciting the fluorescent markers. Illumination light is transmitted to and through an optic lens system 22 which focuses light onto a light pipe 24. The light pipe 24 may create a homogeneous light, which is then transmitted to the fiber optic light guide 26. The light guide 26 may include multiple optic fibers and is connected to a light post 28, which is part of the endoscope 12. The endoscope 12 includes an illumination pathway 12′ and an optical channel pathway 12″.

The endoscope 12 may include a notch filter 131 that allows some or all (preferably, at least 80%) of fluorescence emission light (e.g., in a wavelength range of 830 nm to 870 nm) emitted by fluorescence markers 2 in the target object 1 to pass therethrough and that allows some or all (preferably, at least 80%) of visible light (e.g., in the wavelength range of 400 nm to 700 nm), such as visible illumination light reflected by the target object 1, to pass therethrough, but that blocks substantially all of the fluorescence excitation light (e.g., infrared light having a wavelength of 808 nm) that is used to excite fluorescence emission from the fluorescent marker 2 in the target object 1. The notch filter 131 may have an optical density of OD5 or higher. In some examples, the notch filter 131 can be located in the coupler 13.

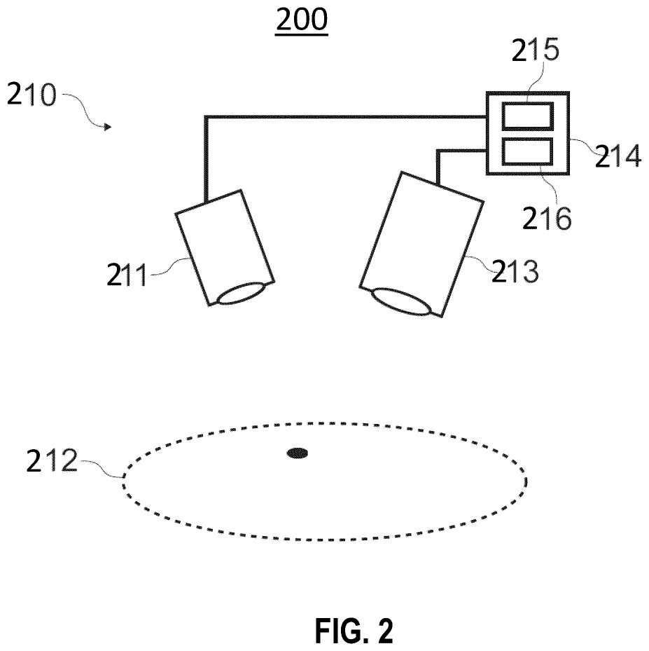

FIG. 2 illustrates an exemplary open field imaging system in accordance with some examples. FIG. 2 illustrates a schematic view of an illumination and imaging system 210 that can be used in open field surgical procedures. As may be seen therein, the system 210 may include an illumination module 211, an imaging module 213, and a video processor/illuminator (VPI) 214. The VPI 214 may include an illumination source 215 to provide illumination to the illumination module 211 and a processor assembly 216 to send control signals and to receive data about light detected by the imaging module 213 from a target 212 illuminated by light output by the illumination module 211. In one variation, the video processor/illuminator 214 may comprise a separately housed illumination source 215 and the processor assembly 216. In one variation, the video processor/illuminator 214 may comprise the processor assembly 216 while one or more illumination sources 215 are separately contained within the housing of the illumination module 211. The illumination source 215 may output light at different waveband regions, e.g., white (RGB) light, excitation light to induce fluorescence in the target 212, a combination thereof, and so forth, depending on characteristics to be examined and the material of the target 212. Light at different wavebands may be output by the illumination source 215 simultaneously, sequentially, or both. The illumination and imaging system 210 may be used, for example, to facilitate medical (e.g., surgical) decision-making, e.g., during a surgical procedure. The target 212 may be a topographically complex target, e.g., a biological material including tissue, an anatomical structure, other objects with contours and shapes resulting in shadowing when illuminated, and so forth. The VPI 214 may record, process, display, and so forth, the resulting images and associated information.

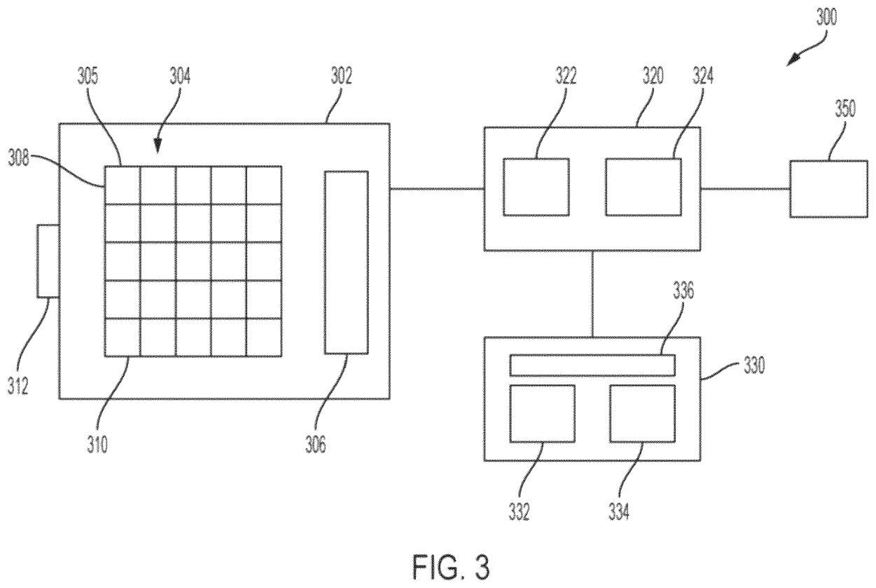

FIG. 3 schematically illustrates an exemplary imaging system 300 that employs an electronic imager 302 to generate images (e.g., still and/or video) of a target object, such as a target tissue of a patient, according to some examples. The imager 302 may be a rolling shutter imager (e.g., CMOS sensors) or a global shutter imager (e.g., CCD sensors, CMOS sensors). System 300 may be used, for example, for the endoscopic imaging system 10 of FIG. 1A. The imager 302 includes a CMOS sensor 304 having an array of pixels 305 arranged in rows of pixels 308 and columns of pixels 310. The imager 302 may include control components 306 that control the signals generated by the CMOS sensor 304. Examples of control components include gain circuitry for generating a multi-bit signal indicative of light incident on each pixel of the sensor 304, one or more analog-to-digital converters, one or more line drivers to act as a buffer and provide driving power for the sensor 304, row circuitry, and timing circuitry. A timing circuit may include components such as a bias circuit, a clock/timing generation circuit, and/or an oscillator. Row circuitry may enable one or more processing and/or operational tasks such as addressing rows of pixels 308, addressing columns of pixels 310, resetting charge on rows of pixels 308, enabling exposure of pixels 305, decoding signals, amplifying signals, analog-to-digital signal conversion, applying timing, readout, and reset signals, and other suitable processes or tasks. Imager 302 may also include a mechanical shutter 312 that may be used, for example, to control exposure of the image sensor 304 and/or to control an amount of light received at the image sensor 304.

One or more control components may be integrated into the same integrated circuit in which the sensor 304 is integrated or may be discrete components. The imager 302 may be incorporated into an imaging head, such as camera head 16 of system 10.

One or more control components 306, such as row circuitry and a timing circuit, may be electrically connected to an imaging controller 320, such as camera control unit 18 of system 10. The imaging controller 320 may include one or more processors 322 and memory 324. The imaging controller 320 receives imager row readouts and may control readout timings and other imager operations, including mechanical shutter operation. The imaging controller 320 may generate image frames, such as video frames from the row and/or column readouts from the imager 302. Generated frames may be provided to a display 350 for display to a user, such as a surgeon.

The system 300 in this example includes a light source 330 for illuminating a target scene. The light source 330 is controlled by the imaging controller 320. The imaging controller 320 may determine the type of illumination provided by the light source 330 (e.g., white light, fluorescence excitation light, or both), the intensity of the illumination provided by the light source 330, and or the on/off times of illumination in synchronization with image sensor shutter operation. The light source 330 may include a first light generator 332 for generating light in a first wavelength and a second light generator 334 for generating light in a second wavelength. In some examples, the first light generator 332 is a white light generator, which may be comprised of multiple discrete light generation components (e.g., multiple LEDs of different colors), and the second light generator 334 is a fluorescence excitation light generator, such as a laser diode.

The light source 330 includes a controller 336 for controlling light output of the light generators. The controller 336 may be configured to provide pulse width modulation (PWM) of the light generators for modulating intensity of light provided by the light source 330, which can be used to manage overexposure and underexposure. In some examples, nominal current and/or voltage of each light generator remains constant, and the light intensity is modulated by switching the light generators (e.g., LEDs) on and off according to a PWM control signal. In some examples, a PWM control signal is provided by the imaging controller 336. This control signal can be a waveform that corresponds to the desired pulse width modulated operation of light generators.

The imaging controller 320 may be configured to determine the illumination intensity required of the light source 330 and may generate a PWM signal that is communicated to the light source 330. In some examples, depending on the amount of light received at the sensor 304 and the integration times, the light source may be pulsed at different rates to alter the intensity of illumination light at the target scene. The imaging controller 320 may determine a required illumination light intensity for a subsequent frame based on an amount of light received at the sensor 304 in a current frame and/or one or more previous frames. In some examples, the imaging controller 320 is capable of controlling pixel intensities via PWM of the light source 330 (to increase/decrease the amount of light at the pixels), via operation of the mechanical shutter 312 (to increase/decrease the amount of light at the pixels), and/or via changes in gain (to increase/decrease sensitivity of the pixels to received light). In some examples, the imaging controller 320 primarily uses PWM of the illumination source for controlling pixel intensities while holding the shutter open (or at least not operating the shutter) and maintaining gain levels. The controller 320 may operate the shutter 312 and/or modify the gain in the event that the light intensity is at a maximum or minimum and further adjustment is needed.

### Techniques for Simulating a Dynamic Fluorescence Response

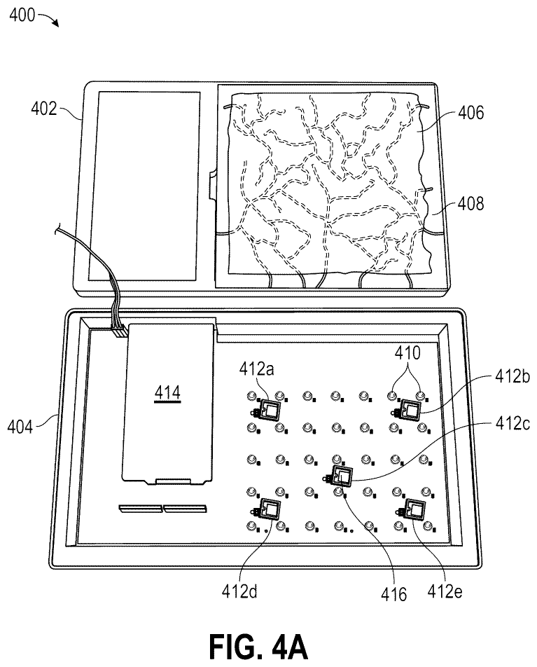

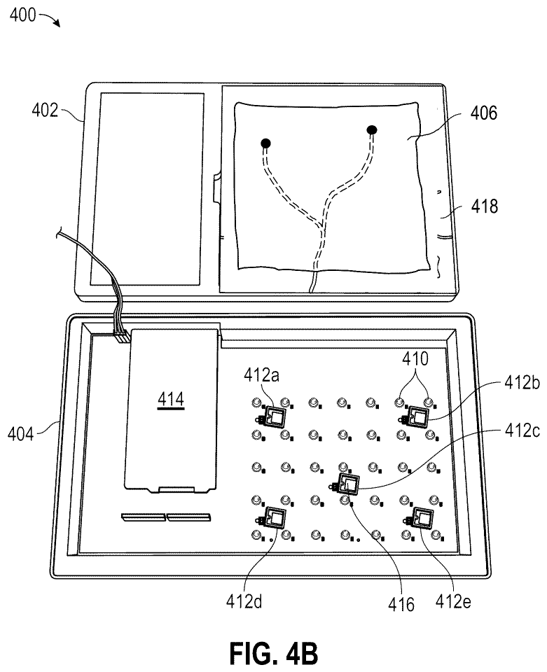

FIGS. 4A and 4B illustrate an exemplary device 400 for simulating a fluorescence response of a tissue (e.g., organic tissue in human and/or animal subjects), in accordance with some examples. The device 400 comprises a top housing component 402 and a bottom housing component 404. In operation, the top housing component 402 is placed on top of the bottom housing component 404 such that the device 400 as a whole can simulate the fluorescence response of a tissue as described herein.

The device 400 comprises a simulated tissue layer in the top housing component 402. In the example depicted in FIG. 4A, the simulated tissue layer includes a simulated vasculature layer 408 and optionally a simulated tissue surface 406 placed over the simulated vasculature layer 408. The simulated tissue layer can mimic the appearance, optical properties, and/or physical properties of organic tissue. The simulated tissue layer of the device can be removed and replaced with a different type of simulated tissue layer. For example, instead of a simulated tissue layer that includes the simulated vasculature layer 408, as shown in FIG. 4A, the device 400 can be used with a simulated tissue layer that includes a simulated lymphatic layer 418, such as shown in FIG. 4B. In other words, the device can work with different types of simulated tissue layers having different materials, appearances, and/or physical structures (e.g., corresponding to different types of tissues, different organs, different vascular structures) to simulate fluorescence responses from different parts of the anatomy depending on the test to be performed.

The optional simulated tissue surface 406 can comprise a layer of material, such as silicone. In one exemplary implementation, the simulated tissue surface 406 can comprise a 0.5 mm layer of silicone with colored pigments.

In the example depicted in FIG. 4A, the simulated vasculature layer 408 is placed beneath the simulated tissue surface 406 to simulate the vasculature and perform the diffusion. The simulated tissue surface 406 can be affixed (e.g., glued) over the simulated vasculature layer 408. In some examples, the simulated tissue surface 406 and the simulated vasculature layer 408 are combined in a single piece of material (e.g., silicone). In the example depicted in FIG. 4B, a simulated lymphatic layer 418 may be placed beneath or combined with the simulated tissue surface 406 instead of a simulated vasculature layer 408 to simulate lymphatic mapping.

In the example depicted in FIG. 4A, the simulated tissue layer comprising the simulated vasculature layer 408 and the simulated tissue surface 406 is placed on the top housing component 402 of the device 400. In some examples, the simulated tissue layer can instead be placed in the bottom housing component 404 over the plurality of illumination sources 410 and the top housing component 406 can close over the bottom housing component 408 in operation. The simulated vasculature layer 408 can comprise a slab of material (e.g., silicone) and includes vessel-like channels on its top surface. In one exemplary implementation, the vessel-like channels have a thickness of 2 mm and a depth of 4 mm to produce a realistic vessel appearance and contrast. The simulated vasculature layer 408 can also be a uniform diffusion layer made up of a slab of material (e.g. silicone). The pigments of the simulated vasculature layer 408 and the simulated tissue surface 406 can be selected to achieve a preferred opacity. In one exemplary implementation, the simulated vasculature layer is 8 mm thick.

In the example depicted in FIG. 4B, the simulated tissue layer comprising the simulated lymphatic layer 418 and the simulated tissue surface 406 can be placed on the top housing component 402 of the device 400, as described above with reference to FIG. 4A. In some examples, the simulated tissue layer can instead be placed in the bottom housing component 404 over the plurality of illumination sources 410 and the top housing component 406 can close over the bottom housing component 408 in operation. The simulated lymphatic layer 418 can comprise a slab of material (e.g., silicone) that includes vessel-like channels and node-like depressions. The simulated lymphatic layer 418 can include a “shield” configured to prevent light from leaking outside of the defined areas of the lymph nodes and vessels simulated by the simulated lymphatic layer 418. The shield can be embedded within the simulated lymphatic layer 418. The shield can comprise polylactic acid (PLA) and can be 3D printed. Optionally, the simulated tissue surface 406 can include an incision point with “flaps” that can be opened to expose a simulated lymph node of simulated lymphatic layer 418 for inspection.

The device 400 further comprises a plurality of illumination sources 410, such as a plurality of LEDs, in the bottom housing component 404. In operation, the top housing component 402, in its depicted orientation, is placed over the bottom housing component 404 in its depicted orientation. Accordingly, the plurality of illumination sources 404 are underneath the simulated vasculature layer 408 (or the simulated lymphatic layer 418), which is underneath the simulated tissue surface 406.

The device 400 further comprises one or more optical detectors 412a-e, such as one or more photodiodes, to detect any fluorescence excitation light incident on the simulated tissue surface 406. The one or more optical detectors 412a-e may be placed in a number of locations in the device 400. In some examples, the one or more optical detectors 412a-e can be placed above the plurality of illumination sources 410. For example, the one or more optical detectors 412a-e may be embedded in the simulated vasculature layer 408 (or the simulated lymphatic layer 418) or placed over the simulated tissue surface 406. In some examples, the one or more optical detectors 412a-e can be in plane with the plurality of illumination sources 410 to prevent any shadow caused by the one or more optical detectors 412a-e to appear in the simulated fluorescence response. The one or more optical detectors 412a-e can be isolated from crosstalk from the plurality of illumination sources 410 via optical filter(s) or mechanical isolation (e.g., being placed over the plurality of illumination sources 410). In some examples, the one or more optical detectors 412a-e include one or more filters such as filter 416. The spectral filters can ensure that the illumination sources emit light only in response to a fluorescence excitation light emitted by the imaging system, and not in response to any visible white-light illumination (e.g., emitted to produce a color image).

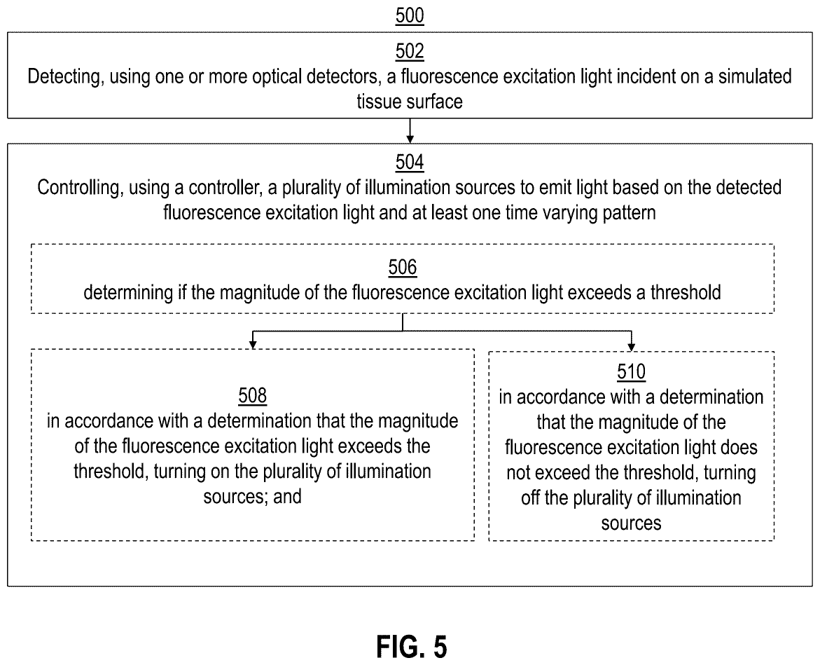

The device 400 further comprises a controller 414 configured to control the plurality of illumination sources 410 to emit light based on the detected fluorescence excitation light and at least one time-varying pattern, as described below with reference to FIG. 5.

FIG. 5 illustrates an exemplary process 500 for simulating a fluorescence response of a tissue, in accordance with some examples. The process 500 may be performed by a system or device such as the device 400 in FIG. 4A or 4B. In some examples, process 500 is performed using a client-server system, and the blocks of process 500 are divided up in any manner between the server and one or more client devices. In some examples, process 500 is performed using only a client device or only multiple client devices. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail below) are exemplary by nature and, as such, should not be viewed as limiting.

At block 502, an exemplary device detects a fluorescence excitation light incident on a simulated tissue surface of the device. The fluorescence excitation light can comprise an infrared light, a visible light, or an ultraviolet light. For example, with reference to FIGS. 4A-4B, the device 400 can detect, using one or more optical detectors 412, a fluorescence excitation light incident on the simulated tissue surface 406. The fluorescence excitation light can serve as a signal to the device to start emitting light to simulate the fluorescence response of a tissue, as described below. Similarly, absence of the fluorescence excitation light can serve as a signal to the device to stop emitting light to simulate the fluorescence response of a tissue, so that the device only emits light while fluorescence excitation light is detected.

At block 504, the device controls a plurality of illumination sources to emit light based on the detected fluorescence excitation light and at least one time-varying pattern. For example, with reference to FIGS. 4A-4B, the device 400 can control, using the controller 414, the plurality of illumination sources 410 to emit light based on the detected fluorescence excitation light incident on the simulated tissue surface 406 and at least one time-varying pattern. The emitted light by the plurality of illumination sources may comprise an infrared light, a visible light, or an ultraviolet light. The system may be fully configurable such that a user may choose a fluorescence response to simulate, as described below.

Block 504 optionally can comprise blocks 506-510. At block 506, the device determines if the magnitude of the fluorescence excitation light exceeds a threshold. In some examples, the threshold can be set as an absolute value. In some examples, the threshold can be set as a relative value of the magnitude of the detected fluorescence excitation light during the “on” time (i.e., during the time period in which the fluorescence excitation light is detected). In other words, the device can dynamically define the threshold based on the magnitude of the detected excitation light over time. In one example the relative threshold is 25% of the detected fluorescence excitation light during the “on” time. In that example, the magnitude of the detected excitation light must drop by 75% for the system to determine that the excitation light is off. This can help to improve the stability of the simulated fluorescence response and reduce false triggering of the device by crosstalk from the plurality of illumination sources. Optionally, the device can decrease the threshold over time if it is not triggered anymore to ensure that the device can start simulating the fluorescence response again if the signal from the fluorescence excitation light reduces significantly.

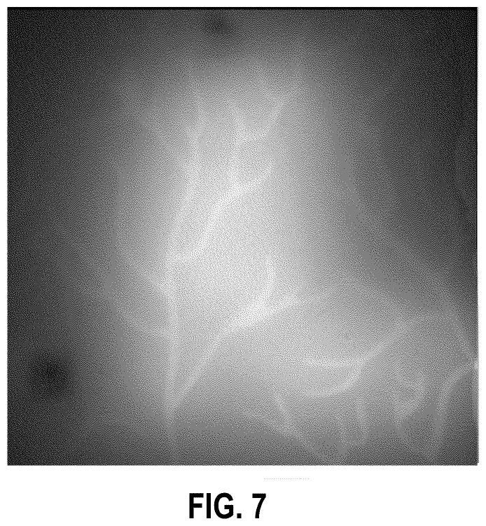

At block 508, in accordance with a determination that the magnitude of the fluorescence excitation light exceeds the threshold, the device turns on the plurality of illumination sources to emit light based on the detected fluorescence excitation light and at least one time-varying pattern. FIG. 7 illustrates a fluorescence response produced by an exemplary device implemented according to the techniques described herein, imaged by a fluorescence imaging system.

On the other hand, at block 510, in accordance with a determination that the magnitude of the fluorescence excitation light does not exceed the threshold, the device can turn off the plurality of illumination sources.

The magnitude of the emitted light can be determined based on the magnitude of the fluorescence excitation light. For example, the magnitude of the emitted light can be proportional to the magnitude of the fluorescence excitation light. Optionally, the magnitude of the emitted light can be scaled. For example, the magnitude of the emitted light is at least partially based on an instantaneous value and a maximum value of the magnitude of the fluorescence excitation light during the on time of the fluorescence excitation light.

$$Laser Intensity ( I ) = \frac{Instantaneous ADC}{Maximum ADC} ,$$

where ADC is representative of the magnitude of the fluorescence excitation light e.g. measured with a photo diode.

Accordingly, the intensity of the emitted light varies to mimic the fluorescence response of a tissue as the distance varies between the excitation light source (e.g., a fluorescence imager) and the tissue. This is desirable because many modern fluorescence imaging systems provide mechanisms to compensate for the variations of fluorescence signal that occur as a result of the variation in working distance. Thus, the techniques described herein can provide a realistic testing and demonstration environment for these imaging systems, allowing any background subtraction and overlay algorithms used by the imaging systems to operate as intended.

The magnitude of the emitted light can be further determined based on at least one time-varying pattern. The time-varying pattern can represent the quantity of fluorescence imaging agent in the model. In some examples, the time-varying pattern can include pulsing between a first intensity level and a second intensity level different from (e.g., lower than) the first. The time-varying pattern can represent various types of fluorescence responses, such as the perfusion growth and decay, the pulsing streak associated with ureter imaging, the steady glow associated with lymph node imaging, etc. For example, the time-varying pattern may comprise a fluorescence growth and decay function, which includes one or more parameters for simulating the ingress and egress of a fluorescent contrast agent at the tissue over time (e.g., the path of brightness). The parameters allow the time-varying pattern to be fully customizable to indicate the desired fluorescence response. In some examples, the time-varying pattern is represented by f(t) and the emitted light is represented as:

$$Emitted Light = f ( t ) \times I$$

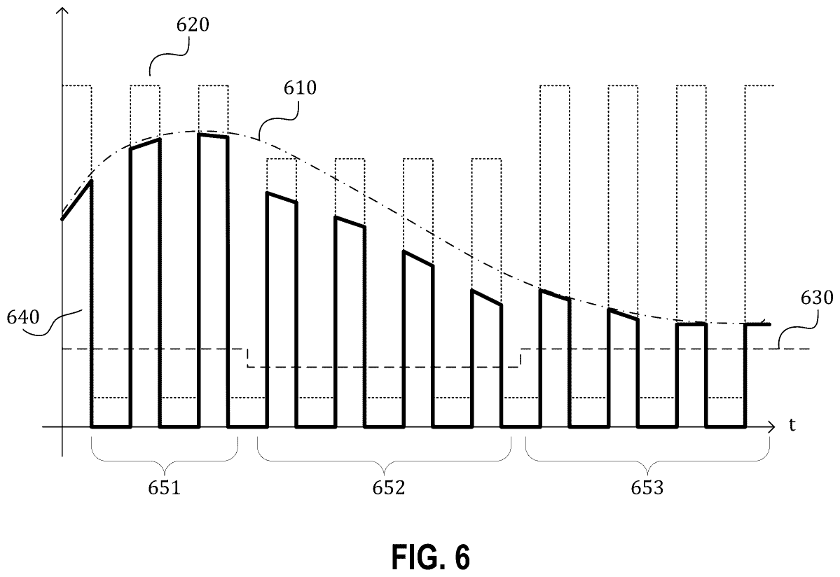

FIG. 6 illustrates an exemplary diagram demonstrating a possible relationship between the magnitude of a fluorescence excitation light and the magnitude of a light emitted by one or more illumination sources of a simulation device, in accordance with some examples. With reference to FIG. 6, the dotted bars 620 represent the magnitude of the fluorescence excitation light as detected by the optical detectors of the simulation device. As described herein, a higher magnitude of the fluorescence excitation light may be indicative of a closer distance between the simulation device and the light source (e.g., an imaging system). As shown, the dotted bars 620 do not go to zero, indicating that the simulation device may always detect some amount of light.

With reference to FIG. 6, the solid bars 640 represent the magnitude of the light emitted by the simulation device. As shown, the magnitude of the emitted light corresponds to a time-varying pattern 610. As described herein, the time-varying pattern may be specific to a single illumination source or multiple illumination sources of the simulation device. Further, the magnitude of the emitted light (as indicated by the solid bars 640) is scaled based on the magnitude of the fluorescence excitation light (as indicated by the dotted bars 620). In a first time period 651 and a third time period 653, the magnitude of the emitted light (as indicated by the solid bars 640) matches the time-varying pattern 610 due to the magnitude of the fluorescence excitation light (as indicated by the dotted bars 620) in the time periods. In contrast, in a second time period 652, the magnitude of the emitted light (as indicated by the solid bars 640) is scaled to be lower than the time-varying pattern 610 due to the magnitude of the fluorescence excitation light (as indicated by the dotted bars 620) in that time period.

FIG. 6 further depicts a line 630 indicative of the threshold for the magnitude of the fluorescence excitation light to turn on the illumination source(s) of the simulation device. As shown, the threshold 630 can be scaled based on the magnitude of the detected fluorescence excitation light. In other words, the threshold 630 can be lower in response to detection of a lower fluorescence excitation light.

It should be appreciated that FIG. 6 is merely exemplary and the present disclosure is not limited as such. For example, the depicted example in FIG. 6 illustrates pulsed light signals, but a simulation device may operate with different types of light signals (e.g., a continuous light signal). Further, FIG. 6 illustrates a possible relationship between the magnitude of the fluorescence excitation light and the magnitude of the light emitted by a simulation device but is not a precise timing diagram.

Optionally, the time-varying pattern is based on a file comprising a plurality of fluorescence image frames. The sequence of fluorescence image frames represents the fluorescence response to be simulated by the device. Given each frame, the device can control the plurality of illumination sources to emit light and the magnitude of the emitted light by each illumination source can be based on the corresponding pixel(s) in the frame. For example, the magnitude of the emitted light can be the product of the detected fluorescence excitation light and the fluorescence signal in the frame as specified in the file. The frame rate at which the device updates the fluorescence response may be triggered by a timer or based on every laser pulse detected. The file can be artificially generated or generated based on a set of fluorescence video data (e.g., recorded real-life scenes from surgical videos).

In one exemplary implementation, the device comprises one or more photodiodes, which can detect a laser pulse associated with the fluorescence excitation light and output a current proportional to the intensity of the laser pulse. An amplifier converts the current to a voltage signal, and an analog-to-digital converter can convert the voltage signal to a 10-bit value representative of the magnitude of the fluorescence excitation light. The device can compare the 10-bit value to a threshold and turn on or off the plurality of illumination sources if the threshold is exceeded. Further, a time-varying pattern may be applied to the emitted light in response to a user input (e.g., a button press). The magnitude or brightness of the emitted light may be controlled via amplitude modulation of the drive current, or by PWM modulation of the drive current (e.g., at a frequency faster than the acquisition rate of the imaging system). On the other hand, if the threshold is not exceeded, the LEDs can be set to low, and the current flow can be restricted.

### Customization

The device described herein is fully customizable and configurable. For example, the device can allow a user to program any aspect of the device, including when the time-varying pattern is to be applied to the emitted light, which time-varying pattern(s) to apply, and whether different time-varying patterns are to be applied to different illumination sources, as described below.

The time to apply the time-varying pattern (i.e., when f(t) is applied to I) can be based on (e.g., responsive to) a user input. As discussed above, the plurality of illumination sources can be turned on or off based on a comparison between the detected fluorescence excitation light and a threshold. While the plurality of illumination sources are turned on, the device can start to apply the time-varying pattern to the emitted light in response to receiving a user input representing the injection of the fluorophore. In some examples, the user can program the device such that the time-varying pattern is automatically applied when the plurality of illumination sources are turned on.

Further, the user can program the device to apply different time-varying patterns to different illumination sources. For example, a first illumination source may be located underneath a first area of the simulated tissue surface corresponding to a first tissue and thus its emitted light needs to simulate the fluorescence response of the first tissue, while a second illumination source may be located underneath a second area of the simulated tissue surface corresponding to a second tissue and thus its emitted light needs to simulate the fluorescence response of the second tissue. Accordingly, the device can be configured to control the first illumination source based on a first time-varying pattern (representing the fluorescence response of the first tissue) and the second illumination source based on a second time-varying pattern (representing the fluorescence response of the second tissue). For example, when a fluorophore is injected, the illumination sources representing large vessels may light up first (e.g., due to the early arrival of the blood) before the illumination sources representing the capillary bed are turned on, and the ingress/egress rates may differ as well.

Further, the user can program the device to specify how signals from the one or more optical detectors can affect the plurality of illumination sources. Optionally, the device comprises multiple optical detectors and can aggregate signals from the multiple optical detectors to determine whether to turn on/off the plurality of illumination sources. Optionally, the signals from different optical detectors or different groups of optical detectors may be used to control different groups of illumination sources. For example, the signals from multiple optical detectors (which are spatially distributed) can be used to calculate an excitation light pattern or distribution. For example, first signals from a first group of optical detectors can be used to emit a light of a first magnitude (calculated based on the first signals) using a first group of illumination sources, while second signals from a second group of optical detectors can be used to emit a light of a second magnitude (calculated based on the second signals) using a second group of illumination sources. As another example, the system may use multiple detectors to apply different intensity scales at different areas, and each illumination source can be controlled independently based on interpolated signals. In other words, an array of illumination sources can be controlled such that each illumination source emits a light proportional to the corresponding pixel in the excitation light pattern or distribution, thus creating an intensity pattern that mirrors the excitation light pattern or distribution. This feature can help to simulate the accurate response for any non-flat illumination field and can be particularly useful for camera systems that have algorithms that compensate for such a non-flat field.

Further, the device can allow the user to select which fluorophore(s) to simulate using the device. The plurality of illumination sources may emit in any spectral band matched to the fluorophore the device is intended to simulate (e.g., based on the user selection). In some examples, the device can support multiple wavelengths at the same time (e.g., using different types of illumination sources side-by-side). Similarly, the optical detectors can be configured to detect the wavelength(s) of interest based on the detected excitation light wavelength(s).

### Additional Optional Components

The device described herein may comprise one or more spectral filters, which can be placed over the one or more optical detectors (e.g., optical detectors 412a-e in FIGS. 4A-4B). The spectral filters can ensure that the illumination sources emit light only in response to a fluorescence excitation light emitted by the imaging system, and not in response to any visible white-light illumination (e.g., emitted to produce a color image).

The device described herein may further comprise a fluorescent dye (e.g., a fluorescent dye with an emission profile around 850 nm) that is excitable by the LED emission. For example, with reference to the example depicted in FIG. 4A, the fluorescent dye may be painted onto the larger vessel-like channels in the simulated vasculature layer such that, to a user of the device, the larger vessels are visualized first followed subsequently by the microvasculature/tissue, thus improving the realism of the simulated fluorescence response. This configuration, when initially illuminated by a dimmer emission from the illumination sources in the device, may allow visualization of the large vessels before the scattered illumination from the illumination sources becomes visible in the rest of the simulated vasculature layer. Optionally, a custom long pass dichroic filter (e.g., on a plastic substrate) can be placed between the LEDs and the simulated vasculature layer (with notches in the filter design for the excitation wavelength(s) so as not to interfere with detection of the imaging device excitation illumination, or alternatively with physical cutouts in the filter to avoid covering the optical detectors), so as to partially suppress the LED emission intensity relative to the painted fluorescent dye emission. Optionally, the device may comprise one or more light pipes embedded in the simulated vasculature layer or underneath it to direct the light from the illumination sources (e.g., toward the vessel-like channels rather than directly upward).

The device described herein may comprise one or more PWM controllers for controlling the plurality of illumination sources. For example, the device can comprise dedicated PWM controllers that allow the control of each LED separately. Some PWM controllers allow specification of a global or semi-global intensity factor and an individual intensity factor. The global or semi-global factor can be used for the intensity control based on the detected excitation light by the optical detectors and the individual intensity factor can be used for simulating the time-varying fluorescence response. This can prevent the use of a microcontroller to calculate the intensity for each illumination source individually.

The device described herein may use a comparator of the controller to determine whether to turn on or off the illumination sources, rather than relying on the ADC values as described above. This can lead to a faster response time to turn on or off the illumination sources, especially if many optical detectors are used in the device. The comparator reference voltage may be adjustable, for example, by way of a global command to the controller (e.g., PWM controller) or a switch that controls the supply of all the illumination sources.

Optionally, the controller comprises a programmable device (e.g., FPGA) for controlling the plurality of illumination sources or generating the PWM signal. Optionally, the controller comprises an adjustable current or voltage source to drive the plurality of illumination sources.

The device described herein may allow a user to control, configure, and/or customize the device via one or more user inputs. The one or more user inputs may comprise one or more button presses, one or more gestural inputs, one or more textual inputs, one or more auditory inputs, one or more selections, or any combination thereof. For example, the user may press one or more buttons built into the device to turn on or off the illumination sources of the device. In another example, the user may use gestures to control the playback of the fluorescence files described herein through gestures such as moving the imaging device from left to right, turning on/off the imaging device within a time period, blocking the fluorescence excitation light for a time period, starting on first laser, turning on/off the laser multiple times, etc. With such a gesture control the device reacts to the user based on the optical detectors without the need for a specific user input. For example, the playback of a fluorescence inflow (simulated injection of the imaging agent) starts a specific time after the system has first detected the excitation light. Another example is that the system would restart the simulation if the excitation light turned on/off at a >20 Hz, <1 Hz interval 2 times. Optionally, the device allows control via a data port (e.g., UART, Ethernet, Wi-Fi) to allow remote controlled operation (e.g., for quality control, automated testing, etc.).

The spatial detail in the emitted fluorescence signals can be affected by the pitch or resolution of the illumination sources. The pitch can be adjusted (e.g., reduced) to increase the spatial detail that can be simulated. Further, the device may comprise a plurality of spatial light modulators to simulate the desired spatial detail.

The techniques described herein may be used in a 3-dimensional simulated tissue body. The 3-dimensional simulated tissue body may comprise a plurality of optical fibers terminating at different locations within the body, which can be dynamically controlled to simulate dynamic fluorescence patterns according to the methods described herein (e.g., in a minimally invasive surgery scenario). The device can be particularly useful for surgical training purposes.

White light visible texture/structure can optionally be added to the simulated tissue surface by printing on it. The printing can be done for example with an inject printer. Depending on the selection of the color/paint, the print can be substantially transparent for the excitation light and the simulated fluorescence light.

The foregoing description, for the purpose of explanation, has been described with reference to specific examples or aspects. However, the illustrative discussions above are not intended to be exhaustive or to limit the invention to the precise forms disclosed. For the purpose of clarity and a concise description, features are described herein as part of the same or separate variations; however, it will be appreciated that the scope of the disclosure includes variations having combinations of all or some of the features described. Many modifications and variations are possible in view of the above teachings. The variations were chosen and described in order to best explain the principles of the techniques and their practical applications. Others skilled in the art are thereby enabled to best utilize the techniques and various variations with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application is hereby incorporated herein by reference.

What is claimed is:

1. A device for simulating a fluorescence response of a tissue, comprising:
a simulated tissue layer;
one or more optical detectors to detect a fluorescence excitation light incident on the simulated tissue layer;
a plurality of illumination sources underneath the simulated tissue layer; and
a controller configured to control the plurality of illumination sources to emit light based on the detected fluorescence excitation light and at least one time-varying pattern.

2. The device of claim 1, wherein the fluorescence excitation light comprises an infrared light, a visible light, or an ultraviolet light.

3. The device of claim 1, wherein the emitted light by the plurality of illumination sources comprises an infrared light, a visible light, or an ultraviolet light.

4. The device of claim 1, wherein controlling the plurality of illumination sources to emit light comprises:
determining if the magnitude of the fluorescence excitation light exceeds a threshold;
in accordance with a determination that the magnitude of the fluorescence excitation light exceeds the threshold, turning on the plurality of illumination sources; and
in accordance with a determination that the magnitude of the fluorescence excitation light does not exceed the threshold, turning off the plurality of illumination sources.

5. The device of claim 4, wherein the threshold is set as an absolute value or as a relative value of the detected fluorescence excitation light during an on time.

6. The device of claim 1, wherein the controller is configured to determine the magnitude of the emitted light based on the magnitude of the fluorescence excitation light incident on the simulated tissue layer of the device.

7. The device of claim 6, further comprising: wherein the magnitude of the emitted light is proportional to the magnitude of the fluorescence excitation light.

8. The device of claim 1, wherein the at least one time-varying pattern comprises pulsing between a first intensity level and a second intensity level lower than the first.

9. The device of claim 1, wherein the at least one time-varying pattern comprises a fluorescence growth and decay function.

10. The device of claim 9, wherein the at least one fluorescence growth and decay function comprises one or more parameters for simulating ingress and egress of a fluorescent contrast agent at the tissue over time.

11. The device of claim 1,
wherein the plurality of illumination sources comprises a first illumination source and a second illumination source,
wherein the at least one time-varying pattern comprises a first time-varying pattern and a second time-varying pattern; and
wherein controlling the plurality of illumination sources of the device to emit light further comprises:
controlling the first illumination source based on the first time-varying pattern; and
controlling the second illumination source based on the second time-varying pattern.

12. The device of claim 1, wherein the at least one time-varying pattern is based on a file comprising a plurality of fluorescence image frames.

13. The device of claim 12, wherein the file is artificially generated or generated based on a set of fluorescence video data.

14. The device of claim 1, wherein the simulated tissue layer comprises silicone.

15. The device of claim 1, wherein the one or more optical detectors comprise one or more photodiodes.

16. The device of claim 1, wherein the plurality of illumination sources comprises a plurality of light-emitting diodes (LEDs).

17. The device of claim 1, further comprising one or more spectral filters over the one or more optical detectors.

18. The device of claim 1, further comprising one or more long pass dichroic filters, one or more light pipes, or any combination thereof.

19. The device of claim 1, wherein the controller comprises one or more PWM controllers for controlling the plurality of illumination sources.

20. The device of claim 1, wherein the controller comprises an adjustable current or voltage source to drive the plurality of illumination sources.

21. The device of claim 1, wherein the controller comprises a programmable device for controlling the plurality of illumination sources.

22. The device of claim 1, further comprising a data port for remote control.

23. The device of claim 1, wherein the device can be wirelessly controlled.

24. The device of claim 1, wherein controlling the plurality of illumination sources of the device to emit light is further based on one or more user inputs.

25. The device of claim 24, wherein the one or more user inputs comprise one or more button presses, one or more gestural inputs, one or more textual inputs, one or more auditory inputs, one or more selections, or any combination thereof.

26. A method for simulating a fluorescence response of a tissue, comprising:
detecting, using one or more optical detectors, a fluorescence excitation light incident on a simulated tissue layer; and
controlling, using a controller, a plurality of illumination sources underneath the simulated tissue layer to emit light based on the detected fluorescence excitation light and at least one time-varying pattern.

27. A non-transitory computer-readable storage medium storing one or more programs, the one or more programs comprising instructions, which, when executed by one or more processors of an electronic device, cause the electronic device to:
detect, using one or more optical detectors, a fluorescence excitation light incident on a simulated tissue layer; and
control, using a controller, a plurality of illumination sources underneath the simulated tissue layer to emit light based on the detected fluorescence excitation light and at least one time-varying pattern.
